(19) United States
(12) Patent Application Publication
(10) Pub. No.: US 2010/0087524 A1
(43) Pub. Date: Apr. 8, 2010

(54) Flavour Improving Substances

(76) Inventors: Stephan Haiber, Naarden (NL); Thorsten Koenig, Naarden (NL); Eric Kohlen, Leusden (NL); Adri De Klerk, Made (NL); Chris Winkel, Bussum (NL)

Correspondence Address: CURATOLO SIDOTI CO., LPA, 24500 CENTER RIDGE ROAD, SUITE 280, CLEVELAND, OH, 44145, US

(21) Appl. No.: 12/528,769

(22) PCT Filed: Nov. 20, 2007
(86) PCT No.: PCT/NL2007/050576

§ 371 Date: Dec. 4, 2009

Related U.S. Application Data
(60) Provisional application No. 60/903,832, filed on Feb. 28, 2007.

(30) Foreign Application Priority Data
Feb. 28, 2007 (EP) 07103219.7

(51) Int. Cl. A61K 31/235; A23L 1/226; A23L 2/56; C07C 69/76; C07C 69/84; A24B 15/30; A61P 43/00

(52) U.S. Cl. 514/533; 426/538; 560/103; 560/76; 560/104; 514/532; 514/544; 131/275; 131/276

(57) ABSTRACT

One aspect of the invention relates to a product selected from the group consisting of foodstuffs, beverages, oral care products, tobacco products and flavourings, said product containing at least 0.01 mg/kg of one or more flavour improving substances selected from the group of substituted phenol esters represented by the following the formula, salts of these compounds and combinations thereof: Formula (I). The invention also relates to novel substituted phenol esters represented by the above formula. Examples of the present flavour improving substances include lactoyl vanillin, lactoyl ethylvanillin, lactoyl isovanillin, lactoyl p-hydroxybenzaldehyde, lactoyl p-hydroxy-m-methoxycinnamaldehyde, lactoyl p-hydroxy-m-methoxycinnamic acid, lactoyl vanillic acid, lactoyl homovanillic acid, lactoyl-m-hydroxybenzoic acid, lactoyl isovanillic acid, lactoyl-p-hydroxy-m-methoxyacetophenone, oleyl vanillin, decanoyl vanillin, divanillyl succinate, lactoyl-o-hydroxy-m-methoxybenzaldehyde, lactoyl eugenol, gluconyl vanillin, and edible salts and esters thereof.

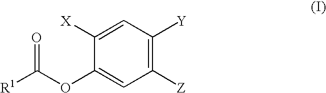

### TECHNICAL FIELD OF THE INVENTION

The present invention relates to flavour improving substances that may suitably be applied in foodstuffs, beverages, oral care products and tobacco products to enhance the flavour thereof. The invention also relates to flavourings, foodstuffs, beverages, oral care products and tobacco products containing such flavour improving substances. Furthermore, the present invention provides a method of flavouring foodstuffs, beverages, oral care products and tobacco products. The present flavour modulating substances can suitably improve and complement the impact of other flavour and/or taste imparting substances

### BACKGROUND OF THE INVENTION

The flavour of foodstuffs and beverages consists of two parts: the aroma and the taste. In general what is perceived through the olfactory epithelium in the nasal cavity is referred to as ‘aroma’, whereas the term ‘taste’ is generally used to describe the sensory impact that is perceived via the mouth, especially the tongue. The flavour sensation experienced upon consumption, especially taste, provides the final analysis of food prior to ingestion thereof. Visual and olfactory (smell) signals already give a first indication but only after intake of the food into the mouth the final decision is made either to ingest or to reject the food. Sweet taste is usually a signal that the food is safe (appetising) leading to ingestion of the food. The ‘reactions’ to salt and umami are really dependent on the strength of the signal. Bitter and sour are usually repulsive taste sensations, leading to rejection. Temperature is another measure by which the food is judged just as well as aching sensations like capsaicin (hot pepper) and certain chemicals (like carbon dioxide).

In short, this means that taste is a very important and very complex system. Until recently most flavour research was focused towards aroma. Especially the last years a series of publications relating to molecules with a (positive) contribution to the taste of foodstuffs has emerged.

Another interesting aspect of taste is that it can have an impact on aroma perception. It was reported that people having artificially sweetened water in their mouth were significantly more sensitive to the smell of benzaldehyde than people having plain water in their mouth (P. Dalton et al, Nature Neurosci. 3, 431-432 (2000)).

Most research on taste modulation so far has been devoted to taste enhancement in savoury products. Several, mainly Japanese, publications describe umami molecules, i.e. alternatives to mono sodium glutamate (MSG) (H Suzuki et al, J Agric Food Chem 50, 313-318 (2002); K Shima et al, J Agric Food Chem 46, 1465-1468 (1998); Y Ueda et al, Biosc Biotech Biochem 61, 1977 (1997)).

In EP 1291342, a ‘general taste enhancer’ is disclosed that was reported to be suitable for enhancing sweetness as well. In patent applications WO 97/04667 and WO 04/075633 tripeptides and amino acid condensates with lactic acid and succinic acid are described that have both their own taste as well as some enhancing properties. Alpha keto acids are reported to give body and mouthfeel to foodstuffs they are added to (U.S. Pat. No. 6,287,620).

Chlorogenic acids are claimed to enhance sweetness and to reduce bitterness (WO 02/100192).

In sweet and beverage products, further examples of the importance of the gustative dimension of flavourings have been reported. These examples include taste attributes such as bitterness, tingling and cooling-freshness.

Similarly, cinnamic aldehyde and eugenol, constituents of cinnamon oil, are used in flavouring composition for confectionary products, not only for their smell but also because they impart a warm and tingling taste. The oral pungency of cinnamic aldehyde was described as burning and tingling by Cliff M and Heymann H (Journal of Sensory Studies 7, 279-290 (1992)). According to the same authors eugenol exhibits a long-lasting numbing effect. Cinnamon oil has been proposed as a taste improving flavouring. International patent application no. WO 90/06689 discloses that cinnamon oil, among other spice extracts, added to a minty flavour formulation, can be used to improve the long-lasting flavour of chewing-gum.

Vanillyl alcohol derivatives (e.g. vanillyl methyl or ethyl ether) are disclosed in patent US 2002/0013235 as having a strong pungent taste and warm feeling imparting effect. The same substances are described in patent JP 57082308 to be effective in increasing the refresh-feeling imparting effect of menthol. Vanillyl ethyl ether is more specifically disclosed as taste improving agent for beer in patent JP 20044229562. Substances from the same group such as vanillyl n-propyl ether are also disclosed in patent JP 57009729 as useful ingredients for salty taste.

Vanillin (4-hydroxy-3-methoxybenzaldehyde) was identified in the 19^{th} century as the major flavour compound in cured vanilla beans. Many syntheses have been developed for the preparation of vanillin, because vanilla extract is an expensive ingredient. Also substances with a similar flavour profile as vanillin have been identified and manufactured. Ethyl vanillin is an example of such a vanillin-like substance. Ethyl vanillin is about 4 times as strong as vanillin, but it has a more smoky character.

In addition to vanillin the flavour industry uses many other flavour ingredients to compound a good vanilla flavour. During the last decades several FEMA GRAS materials have been developed by the flavour industry to give specific profiles to vanilla flavours. Recent examples are vanillin isobutyrate (FEMA 3754), vanillyl butyl ether (FEMA 3796), vanillyl ethyl ether (FEMA 3815), vanillin 3-(1-menthoxy)propane-1,2-diol acetal (FEMA 3904), vanillin propylene glycol acetal (FEMA 3905) and vanillin butan-2,3-diol acetal (FEMA 4023).

Dikusar et al. (Russian J. of Appl Chem. vol. 78 no. 1 (2005), 120-124) describe the preparative synthesis of a number of vanillin and ‘vanillal’ alkanoates. This article describes the synthesis and chemical/physical properties of vanillin esters wherein the alkanoate is methyl (b), ethyl (c), propyl (d), isopropyl (e), butyl (f), isobutyl (g), pentyl (h), hexyl (i), heptyl (j), dodecyl (k), CH₂Cl (l) and 4-MeC₆H₄O(CH₂)₂ (m). In the article the flavour characteristics of the vanillin esters (b)-(g) are described. It is concluded by the authors that the vanillin and ‘vanillal’ alkanoates are of interest as components of flavour food additives with popular chocolate, caramel, and milk cream smells.

The present invention is concerned with providing new substances having a positive contribution to the flavour, especially taste, of foodstuffs, beverages, oral care products and tobacco products they are incorporated in. More in particular it aims to provide flavour ingredients that can suitably improve the quality of a consumable product, on the one hand by having appreciable flavour and/or taste attributes of its own, especially attributes complementing and/or resembling vanilla flavours, and on the other hand by improving, especially enhancing, the impact of other flavour and/or taste imparting substances, especially sweet taste imparting substances.

### SUMMARY OF THE INVENTION

Unexpectedly, the inventors have found that the quality of flavourings can be improved significantly by incorporating therein an ester of vanillin and a hydroxyl- or carboxyl-substituted C₂-C₇ acid or an ester of vanillin and a C₈-C₂₂ fatty acid. The incorporation of these vanillin esters in consumable products such as foodstuffs, beverages, oral care products and tobacco products provides more body and taste. More in particular, the use of the present flavour improving substances was found to yield on the one hand a flavour character close to natural vanilla extract, especially when used in combination with vanillin, while on the other hand they were found to significantly modify and improve the impact of other flavour and/or taste imparting substances, especially sweet flavour and/or taste imparting substances. Furthermore, these flavour improving vanillin esters were found to reduce the bitter character of vanillin. Unlike vanillin, these vanillin esters do not suffer from a powdery character.

EP 0 470 765 and EP 0 470 766 disclose vanillin-release ester compounds as flavourant release additives for tobacco products that undergo pyrolisis or thermolysis at temperatures of 200-300° C. to release vanillin. Examples of such vanillin esters include di-vanillyl oxalate, di-vanillyl succinate, di-vanillyl glutarate and 4′-formyl-2′-methoxyphenyl 5-hydroxy-2-isopropylhexanoate. There is no indication in either one of these documents that these esters themselves would provide any suitable contribution to the flavour of the tobacco products or that they would have any modulating effects on flavour impact.

Besides vanillin, a number of other, structurally closely related phenol derivatives are widely used in the flavour and fragrance industry. Examples include ethyl vanillin, p- and m-hydroxy benzoic acid, isovanillic acid, p-hydroxy-m-methoxy-acetophenon, eugenol and guaiacol. The inventors have found that esters of these phenols and hydroxyl/carboxyl-substituted C₂-C₇ acids or C₈-C₂₂ fatty acid can also advantageously be applied as flavour improving substances in flavourings, foodstuffs, beverages, oral care products and tobacco products. Thus, the present invention encompasses the use of the aforementioned phenyl esters as flavour improving substances in flavourings, foodstuffs, beverages, oral care products and tobacco products.

### DETAILED DESCRIPTION OF THE INVENTION

Accordingly, one aspect of the invention relates to a product selected from the group consisting of foodstuffs, beverages, oral care products, tobacco products and flavourings, said product containing one or more flavour improving substances selected from the group of compounds represented by the following formula (I), salts of these compounds and combinations thereof:

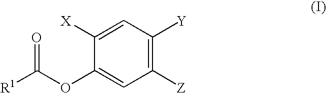

wherein:
X and Z independently represent —H, —OCH₃, —OCH₂CH₃, —CH═O or —COOH;
Y represents —H, —CHO, —COOH, —COCH₃, —CH═CH₂, —CH₂—COOH, —CH₂—CH′CH₂, —CH═CH—CH═O, or —CH═CH—COOH;
at least one of X, Y and Z does not represent —H;
R¹ represents (i) a C₁-C₆ alkyl or C₂-C₆ alkenyl, carrying at least one substituent selected from —OH, —NH₂ and —COOR² and optionally further substituents or (ii) an optionally substituted C₇-C₂₁ alkyl, C₇-C₂₁ alkenyl, C₇-C₂₁ alkadienyl or C₇-C₂₁ alkatrienyl;
R² represents —H or a residue represented by the following formula (II):

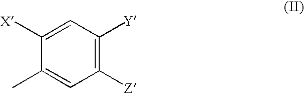

B′, D′ and E′ in formula (II) independently have the same meaning as B, D and E, respectively in formula (I); and
at least one of B′, D′ and E′ does not represent H.

A particularly preferred embodiment of the invention concerns the above-defined products wherein
X and Z independently represent —H, —OCH₃, —OCH₂CH₃ or —COOH;
Y represents —H, —CHO, —COOH, —COCH₃, —CH═CH₂, —CH₂—CH′CH₂ or —CH═CH—COOH;
at least one of X, Y and Z does not represent —H; and
R¹ represents (i) a C₁-C₆ alkyl or C₂-C₆ alkenyl, carrying at least one substituent selected from —OH and —COOR² and optionally further substituents or (ii) an optionally substituted C₇-C₂₁ alkyl, C₇-C₂₁ alkenyl, C₇-C₂₁ alkadienyl or C₇-C₂₁ alkatrienyl.

It is furthermore particularly preferred in accordance with these embodiment, that, in case the product is a tobacco product, Y does not represent —CHO. Still more preferably, in case the product is a tobacco product or a tobacco flavouring, R¹ does not represent —COOR² or C₁-C₆ alkyl carrying a substituent selected from —COOR² and —OH

The term “edible salt”, as used herein, refers to a salt that is generally considered suitable for human consumption, particularly a non-toxic salt. Acceptable salts include base addition salts and acid addition salts of the corresponding free acid. These salts typically may be prepared by conventional means from the free acid of the present flavour modulating substances. Illustrative base addition salts include metallic salts and organic salts. Metallic salts include alkali metal salts, such as sodium and potassium salts, alkaline earth metal salts, such as calcium and magnesium salts. Organic salts include salts made from secondary, tertiary and quaternary amines such as diethylamine, triethylamine, ethanolamine, diethanolamine, and salts made from cationic amino acids such as arginine, lysine and histidine. Examples of suitable acid addition salts include hydrochloride, phosphate, hydrogen phosphate, acetate, aspartate, ascorbate, citrate, gluconate, lactate, succinate, tartrate, etc.

The present inventors have found that the above-mentioned flavour modulating substances are very useful flavour ingredients which, particularly in the presence of other flavour and/or taste imparting substances, are capable of imparting highly appreciated taste sensations to the products in which they are incorporated. Because of this, the present substances can be employed to improve the flavour, especially taste (including “mouthfeel”), of foodstuffs, beverages, oral care products and tobacco products.

The flavour modulating substances of the present invention as such are capable of imparting highly desirable taste attributes. In addition, it has been found that the flavour modulating substances according to the invention are capable of complementing and modifying the sensory impact of other, flavour and/or taste imparting, substances, contained in the aforementioned products, including complementing and modulating , especially enhancing, “vanilla flavour character” and/or “sweet taste impact”.

Throughout this document the term “flavour” is used to describe the sensory impact that is perceived via the mouth, especially the tongue, and the olfactory epithelium in the nasal cavity. The term “complementing and modifying the sensory impact” as used herein refers to the capability of the present compositions or substances to alter the taste and/or aroma impact of other, flavour and/or taste imparting, substances present within the same product, with the proviso that this change in taste impact is not caused by the flavour contribution of said composition or substance per se, but instead that it mainly results from the combined effect of on the one hand the present flavour modulating composition or substance and on the other hand the other flavour and/or taste imparting substance(s). The present flavour modulating substances combine the capability of modulating the taste and/or aroma of other, flavour and/or taste imparting, substances and a taste contribution of their own. The favourable impact of the present flavour modulating substances is believed to be the result of the combination of these two effects.

Because the flavour modulating substances according to the invention are not particularly volatile, they do not produce a strong aroma impact, even though they can affect the aroma impact of other flavour and/or taste imparting substances. Here the term “aroma” refers to the aspect of flavour that is perceived through the olfactory epithelium. Because of the low volatility of the present flavour modulating substances it is believed that the advantageous properties of these substances are somehow associated with the impact that these substances have on the sensory receptors located within the mouth.

Preferably, the products of the invention contain at least at least 0.01 mg/kg, more preferably 0.1 mg/kg, most preferably at least 0.5 mg/kg of the flavour improving substances according to the present invention. According to a particularly preferred embodiment, the product of the present invention is selected from the group consisting of foodstuffs and beverages. Examples of foodstuffs in which the benefits of the present invention can be realised include ice cream, desserts, chocolate, confectionary and bakery custards. Examples of beverages in which the flavour improving substances of the present invention can advantageously be employed include cola, milk, milks shakes and yoghurt drinks. In foodstuffs and beverages the present taste improving substances are typically applied in a concentration that does not exceed 500 mg/kg. Preferably, said concentration does not exceed 100 mg/kg. The term ‘tobacco products’ as used herein refers to any type of tobacco product for smoking as well as for non-smoking applications. It is furthermore noted that tobacco-like products are available for both smoking and non-smoking applications. The use of the present flavour modulating substances in tobacco substitutes is also encompassed by the present invention.

In a particularly preferred embodiment, the present products comprise sweet taste imparting substances, especially natural or artificial sweeteners. Suitable examples of natural sweeteners include sucrose, fructose, glucose, high fructose corn syrup, xylose, arabinose, and rhamnose, the sugar alcohols erythritol, xylitol, mannitol, sorbitol, and inositol. Suitable examples of artificial sweeteners include AceK, aspartame, sucralose, and saccharine. These sweet taste imparting substances may be included in any suitable amount. Since the sweet taste imparting effect of these substances will be enhanced by the presence of the flavour modulating substances of the invention, said amounts may typically be lower than those conventionally applied. Typically an amount will be included which is equivalent in sweetness to at least 0.5 wt % sucrose, preferably 0.5-15 wt %, more preferably 1.0-7 wt %.

According to a preferred embodiment, in formula (I) R¹ represents a C₂-C₆ alkyl or C₂-C₆ alkenyl carrying one or more substituents, said one or more substituents being selected from OH and COOR². Even more preferably, R¹ represents (i) a hydroxyl-substituted, C₂-C₅ alkyl, optionally carrying 1-4 further hydroxyl substituents or (ii) a —COOR²-substituted C₂-C₅ alkyl or a —COOR²-substituted C₂-C₅ alkenyl, optionally carrying 1-3 further —COOR² substituents. Preferred examples of hydroxyl-substituted alkyl residues include —CH(OH)CH₃ and —[CH(OH)]₄CH₂(OH), the former being most preferred. Preferred examples of —COOR²-substituted alkyl or alkenyl residues include —CH₂—CH₂—COOR² and —CH═CH—COOR². R² preferably represents H or a residue as represented in formula (II) wherein X′═X, Y′═Y and Z′═Z. According to another preferred embodiment, in formula (I), R¹ represents (i) a C₁-C₆ alkyl or C₂-C₆ alkenyl, carrying at least one substituent selected from —OH, —NH₂ and —COOR² and optionally further substituents or (ii) C₇-C₂₁ alkyl, C₇-C₂₁ alkenyl, C₇-C₂₁ alkadienyl or C₇-C₂₁ alkatrienyl each optionally substituted with one or more substituents selected from —OH, ═O, C₁-C₄ alkoxyl, —COOH and —NH₂. More preferably, R1 represents an unsubstituted C₇-C₂₁ alkyl, an unsubstituted C₇-C₂₁ alkenyl, an unsubstituted C₇-C₂₁ alkadienyl or an unsubstituted C₇-C₂₁ alkatrienyl. Even more preferably, R¹ represents an unsubstituted C₁₅-C₁₇ alkyl, an unsubstituted C₁₅-C₁₇ alkenyl, an unsubstituted C₁₅-C₁₇ alkadienyl or an unsubstituted C₁₅-C₁₇ alkatrienyl. Most preferably, R¹ represents C₁₇ alkenyl, C₁₇ alkadienyl or C₁₇ alkatrienyl, especially the aliphatic residue of oleic acid, linoleic acid or linolenic acid.

The flavour improving substances of the present invention are preferably represented by formula (I) in which X represents —H or —OCH₃. Examples of such substances include esters of benzoic acid, isovanillic acid and vanillin. Most preferably, X represents —OCH₃.

As explained herein before, the flavour improving substances of the present invention include esters of e.g. m-hydroxy benzoic acid and isovanillic acid. The latter phenols differ from e.g. vanillin in that their phenyl-ring contains an additional carboxyl radical in the Z-position. The benefits of the present invention are particularly manifest in flavour improving substances, such as vanillin, in which Z in formula (I) represents —H.

According to another preferred embodiment, the present flavour improving substances are represented by formula (I) wherein Y represents —CHO, —COOH or —COCH₃. Examples of phenols in which Y represents one of the aforementioned radical include vanillin (—CHO), p-hydroxybenzoic acid (—COOH), isovanillic acid (—COOH) and p-hydroxy-m-methoxy acetophenon (—COCH₃). Most preferably, Y in formula (I) represents —CHO.

In accordance with another preferred embodiment of the invention the flavour improving substances are selected from the group of lactoyl vanillin, lactoyl ethylvanillin, lactoyl isovanillin, lactoyl p-hydroxybenzaldehyde, lactoyl p-hydroxy-m-methoxycinnamaldehyde, lactoyl p-hydroxy-m-methoxycinnamic acid, lactoyl vanillic acid, lactoyl homovanillic acid, lactoyl-m-hydroxybenzoic acid, lactoyl isovanillic acid, lactoyl-p-hydroxy-m-methoxyacetophenone, oleyl vanillin, decanoyl vanillin, divanillyl succinate, lactoyl-o-hydroxy-m-methoxybenzaldehyde, lactoyl eugenol, gluconyl vanillin, and edible salts and esters thereof, most preferably from lactoyl vanillin, lactoyl isovanillin, lactoyl p-hydroxybenzaldehyde, lactoyl p-hydroxy-m-methoxycinnamic acid, lactoyl isovanillic acid, oleyl vanillin, decanoyl vanillin, divanillyl succinate, lactoyl eugenol, gluconyl vanillin, and edible salts and esters thereof.

The inventors have observed that the combined use of vanillin and a flavour improving substance according to the present invention yields particularly good results. Accordingly, the present product, besides the flavour improving substance, advantageously contains at least 0.5 mg/kg, preferably at least 3 mg/kg of vanillin. The combination of vanillin and a flavour improving substance of the present invention was found to produce particularly good flavour results if the flavour improving substance used was a vanillin ester, i.e. a flavour improving substance in which X, Y and Z in formula (I) represent OCH₃, CHO and H, respectively.

In accordance with another aspect of the invention, the product according to the present invention is a flavouring composition containing at least 0.1 wt. %, preferably at least 1 wt. % of one or more flavouring substances and at least 10 mg/kg of a flavour improving substance as defined herein before. The term “flavouring substance” as used herein encompasses any food grade substance that is capable of imparting a detectable flavour impact, typically at concentrations below 1 wt. %, more preferably below 0.1 wt. %. Suitable examples of flavouring substances include alcohols, aldehydes, ketones, esters, ethers, acetates, nitriles, terpenes, hydrocarbons, nitrogenous or sulphurous heterocyclic compounds and essential oils, and said flavouring substances can be of natural or synthetic origin. Many of these are listed in reference texts such as the book by S. Arctander, Perfume and Flavor Chemicals, Montclair, N.J., USA or in other works of a similar nature, as well as in the abundant patent literature in the field of flavours.

Typically, in the present flavour composition the taste modulating substances and flavouring substances as defined herein before are employed in a weight ratio of less than 1:50, preferably less than 1:100, more preferably less than 1:1,000 and most preferably in a weight ratio of less than 1:10,000.

The flavour composition according to the present invention may suitably be prepared in the form of a liquid, a paste or a powder. In a particularly preferred embodiment the flavour composition is a free flowing powder. Typically the present flavouring compositions further comprise at a flavour carrier material. As used herein, the term flavour “carrier material” encompasses a bulk material that is practically neutral from a flavour point of view, i.e. which does not significantly alter the organoleptic properties of flavouring ingredients. Said carrier may be a liquid or a solid. Suitable examples of liquid carriers include emulsifying systems, i.e. a solvent and a surfactant system, or a solvent commonly used in flavours. Suitable non-limiting examples of solvents commonly used in flavours include compounds such as propylene glycol, triacetine, triethyl citrate, benzylic alcohol, ethanol, vegetable oils or terpenes. Examples of solid carriers include absorbing gums or polymers or encapsulating materials.

Examples of such materials, for example, may comprise wall-forming and plasticizing materials, such as mono, di- or trisaccharides, natural or modified starches, hydrocolloids, cellulose derivatives, polyvinyl acetates, polyvinylalcohols, proteins or pectins, maltodextrins or the materials cited in reference texts such as H. Scherz, Hydrokolloids: Stabilisatoren, Dickungs- and Gehermittel in Lebensmittel, Band 2 der Schriftenreihe Lebensmittelchemie, Lebensmittelqualitat, Behr's VerlagGmbH & Co., Hamburg, 1996.

Advantageously, the flavour improving substance is contained in the present flavouring composition in a concentration of at least 40 mg/kg, more preferably of at least 100 mg/kg. Usually, the concentration of the favour improving substance in the present flavour composition does not exceed 10,000 mg/kg.

As explained herein before the flavour improving substances according to the present invention are advantageously applied in combination with vanillin. Hence, according to a particularly preferred embodiment, the present flavour composition contains at least 50 mg/kg, preferably at least 100 mg/kg and most preferably at least 300 mg/kg of vanillin.

In a particularly preferred embodiment, the present flavour compositions comprise sweet taste imparting substances, especially natural or artificial sweeteners, as explained above. These sweet taste imparting substances may be included in any suitable amount.

Since the sweet taste imparting effect of these substances will be enhanced by the presence of the flavour modulating substances of the invention, said amounts may typically be lower than those conventionally applied. Typically an amount will be included which is equivalent in sweetness to at least 2.5wt % of sucrose, preferably 2.5-50, more preferably 5-25 wt %.

Another aspect of the invention relates to a method of preparing a product selected from the group consisting of foodstuffs, beverages, oral care products and tobacco products, said method comprising incorporating in the product at least 0.01 wt. % of a flavouring composition as described herein before. Typically, such method include the addition of from 0.03-5 wt. % of said flavouring composition.

Yet another aspect of the invention concerns the use of a flavour improving substance as defined herein before for improving the flavour of a foodstuff, a beverage or a tobacco product. In an even more preferred embodiment of the invention the use of a flavour improving substance as defined herein before is provided for modulating, especially enhancing, vanilla flavour character and/or sweet taste impact.

The present invention also provides novel flavour improving substances that are represented by the following formula (III) or salts thereof:

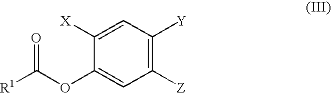

wherein:
X and Z independently represent —H, —OCH₃, —OCH₂CH₃, —CH═O or —COOH;
Y represents —H, —CHO, —COOH, —COCH₃, —CH═CH₂, —CH₂—COOH, —CH₂—CH═CH₂, —CH═CH—CH═O, or —CH═CH—COOH;
at least one of X, Y and Z does not represent —H;
R′ represents
   C₁-C₆ alkyl carrying at least one —NH₂ group and optional further substituents
   —CH(OH)CH₃,
   —[CH(OH)]₄CH₂(OH),
   —CH₂—CH₂—COOR²,
   —CH═CH—COOR² or
   unsubstituted C₁₅-C₂₁ alkyl, unsubstituted C₁₅-C₂₁ alkenyl, unsubstituted C₁₅-C₂₁ alkadienyl or unsubstituted C₁₅-C₂₁ alkatrienyl, preferably C₁₇ alkenyl, C₁₇ alkadienyl or C₁₇ alkatrienyl, each optionally substituted with one or more substituents selected from —OH, ═O, C₁-C₄ alkoxyl, —COOH and —NH₂;
R² represents —H or a residue represented by the following formula (IV):

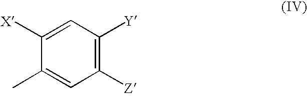

X′, Y′ and Z′ in formula (IV) independently have the same meaning as X, Y and Z, respectively in formula (III); and
at least one of X′, Y′ and Z′ does not represent H.

Particularly preferred are novel flavour improving substances that are represented by the following formula (III) or salts thereof as defined above wherein:
X and Z independently represent —H, —OCH₃ or —OCH₂CH₃;
Y represents —H, —CHO, —COOH or —COCH₃;
at least one of X, Y and Z does not represent —H;
R¹ represents
   —CH(OH)CH₃,
   —[CH(OH)]₄CH₂(OH),
   —CH₂—CH₂—COOR²,
   —CH═CH—COO R² or
   unsubstituted C₁₅-C₂₁ alkyl, unsubstituted C₁₅-C₂₁ alkenyl, unsubstituted C₁₅-C₂₁ alkadienyl or unsubstituted C₁₅-C₂₁ alkatrienyl.

In accordance with these aspects of the invention it is particularly preferred that, in case Y represents —CHO, R¹ does not represent —CH₂—CH₂—COOR².

According to a particularly preferred embodiment novel flavour modulating substances are provide as defined herein before, wherein X represents —H, —OCH₃, —CH═O or —OCH₂CH₃; Y represents —H, —CHO, —COOH, —COCH₃, —CH₂—COOH or —CH═CH—CH═O; and Z represent —H, —CH═O or —COOH. Still more preferably novel flavour modulating substances as defined herein before are provided wherein X represents —H, —OCH₃ or —OCH₂CH₃; Y represents —H, —CHO, —COOH or —COCH₃ and Z represents —H or —COOH.

Most preferably, in the aforementioned formula (III) X represents OCH₃. According to another preferred embodiment, Z represents —H. Likewise, it is strongly preferred that Y in formula (III) represents CHO.

R² preferably represents H or a residue as represented in formula (IV) wherein X′═X, Y′═Y and Z′═Z.

According to another particularly preferred embodiment, the flavour improving substance is a lactic acid ester of a phenolic substance. Accordingly, R¹ advantageously represents —CH(OH)CH₃ Most preferably, the flavour improving substance of the present invention is O-lactoyl vanillin, i.e. a substance according to formula (III) in which X═—OCH₃, Y=—CHO, Z═—H and R¹═—CH(OH)CH_{3.}

It is within the skills of the trained professional to design synthesis routes suitable to prepare any of the novel substances provided by the present invention. Different routes may be available for producing a given substance, some of which may be more convenient and efficient than others. As a general guidance several routes have been illustrated in the examples hereafter. As will be understood by the skilled person based on these examples, the synthesis will typically involve a coupling reaction between a phenol derivative and an aliphatic acid derivative. Preferably an activated ester is reacted with a phenolic hydroxyl group. If a hydroxyl-substituted acid derivative is used, preferably said hydroxyl group is protected by a protecting group, suitable examples of which are known by the skilled person. The reaction conditions are preferably chosen such as to protect any other functionality present in the derivatives to be coupled. In some cases enzymes may be used for catalysing the coupling reaction. Additional steps for introducing or modifying one or more functionalities may be performed before or after said coupling reaction, although before is preferred.

The invention is further illustrated by means of the following non-limiting examples.

### EXAMPLES

### Example 1

### 2-hydroxy-propionic acid 4-formyl-2-methoxy-phenyl ester (lactoyl vanillin) was Prepared as Follows:

### Step 1: Preparation of 5-methyl-1,3-dioxolane-2,4-dione

A solution of a diphosgene (58.8 g; 0.3 mol) in THF (100 mL) was added dropwise to a stirred solution of anhydrous lactic acid (22.5 g; 0.25 mol) in 400 mL of THF at room temperature. The mixture was refluxed for 2 hours. Activated charcoal (5 g) was added (to destroy excess diphosgene) and refluxing was continued for another hour. The mixture was allowed to stand overnight at room temperature. The mixture was filtered and the filtrate was concentrated under reduced pressure to give a viscous oil and solidified by cooling to −78° C. The solids were washed with diethyl ether, filtered and dried in a vacuum oven at 50° C./20 mbar to yield 15 g (22%) of a grey solid. NMR analysis indicated a purity of 90%.

### Step 2: Preparation of lactoyl vanillin

Vanillin (0.75 g; 5 mmol) and 5-methyl-1,3-dioxolane-2,4-dione (0.7 g; 6 mmol) were dissolved in 25 mL of dichloromethane. Three drops of triethyl amine were added carefully. The reaction was slightly exothermic and release of carbon dioxide was fast. The mixture was stirred for an hour at room temperature. The solvent was removed at 40° C./20 mbar. Diethyl ether (25 mL) was added to crystallize the product. Solids were filtered, washed once with diethyl ether and dried to yield 1 g (90%) lactoyl vanillin. NMR analysis indicated a purity of 92%.

### Example 2

Four different vanilla flavoured milk drinks were prepared by mixing the ingredients as given in the following table.

| Ingredients | Milk drink A | Milk drink B | | Milk drink C | | Milk drink D | |
|---|---|---|---|---|---|---|---|
| 1.5% fat | 95 (g) | 95 | (g) | 95 | (g) | 95 | (g) |
| pasteurized | | | | | | | |
| milk | | | | | | | |
| Sugar | 5 (g) | 5 | (g) | 5 | (g) | 5 | (g) |
| Lactoyl vanillin | | 0.5 | (mg) | 1.0 | (mg) | 0.5 | (mg) |
| Vanillin | | | | | | 0.5 | (mg) |

All milk drinks were tasted and evaluated by a sensory panel. Product A was described as sweet milk. Product B was described as sweet, creamy, vanilla. Product C was described as sweet, creamy, vanilla, white chocolate like. Product D was described as vanillin, creamy, vanilla bean like, slightly alcoholic.

### Example 3

Four different vanilla flavoured ice creams were prepared by mixing the ingredients as given in the following table.

| Ingredients | Ice cream A | Ice cream B | | Ice cream C | | Ice cream D | |
|---|---|---|---|---|---|---|---|
| Ice cream, 8% | 100 (g) | 100 | (g) | 100 | (g) | 100 | (g) |
| vegetable fat | | | | | | | |
| Lactoyl vanillin | | 1 | (mg) | 1 | (mg) | | |
| Vanillin | | | | 1 | (mg) | 1 | (mg) |

All ice creams were tasted and evaluated by a sensory panel. Product A was described as sweet and bland. Product B was described as creamy, vanilla. Product C was described as vanillin, sweet, vanilla bean like and long lasting. Product D was described as vanillin, sweet, powdery.

### Example 4

### Preparation of Lactoyl isovanillic acid

0.75 g (5 mmol) of isovanilline was dissolved in 25 mL of dichloromethane. To this solution was added 0.7 g (6 mmol) of 5-methyl-1,3-dioxolane-2,4-dione. To this homogeneous solution were added three drops of triethylamine. A little exothermic effect and a vigorous release of carbon dioxide was observed. After 30 minutes at room temperature solvent was evaporated at 40° C./20 mbar. Diethylether was added to the residue and the product crystallized. Solids were filtered, washed once with diethyl ether and dried in the vacuum oven at 50° C/10 mbar to yield 0.5 g of pure lactoyl isovanillic acid.

### Example 5

### Preparation of Lactoyl 4-hydroxy-3-methoxy-acetophenone

0.4 g (2.4 mmol) of 4-hydroxy-3-methoxyacetophenone was added to 20 mL of methylenechloride. To this solution was added 0.3 g (2.6 mmol) of 5-methyl-1,3-dioxalane-2,4-dione. A few drops of triethylamine were added and a vigorous release of carbon dioxide was observed. Stirring was continued for 30 minutes at room temperature. Solvent was evaporated and the residue was crystallized by adding some diethyl ether. Solids were filtered and dried in the vacuum oven at 60° C/10 mbar to yield 0.3 g of pure lactoyl 4-hydroxy-3-methoxy-acetophenone.

### Example 6

### Preparation of Lactoyl 3-hydroxybenzoic acid

0.41 g (3 mmol) of 3-hydroxybenzoic acid was added to 20 mL of methylenechloride. To this suspension was added 0.3 g of triethylamine and some THF to dissolve everything.

To this solution was added 0.35 g (3 mmol) of 5-methyl-1,3-dioxalane-2,4-dione. No release of carbon dioxide was observed. Stirring was continued for 30 minutes at room temperature. Solvent was evaporated at atmospheric pressure and the residue was taken up in mtbe and washed twice with a diluted hydrochloric acid solution. Organic layer was separated and dried with magnesium sulphate, filtered and evaporated to yield 0.2 g of pure lactoyl 3-hydroxybenzoic acid.

### Example 7

### Preparation of divanillyl succinate

77 g (0.5 mol) of vanillin and 51 g (0.5 mol) of triethyl amine, dissolved in 200 ml of diethyl ether, was added slowly to a solution of 39 g (0.25 mol) of succinyl dichloride dissolved in 150 ml of diethyl ether. The mixture was heated under reflux for about 2 hrs. A brown precipitate was formed. The diethyl ether solution was filtered and the residue was washed with 150 ml of a 10% NaHCO₃-solution. Then the pH was adjusted to 3 by adding a 10% HCl solution. The brown residue was filtered and washed with 2 times 50 ml of cold water and with 2 times 50 ml of diethylether. The crude product was recrystallised twice from ethyl acetate/hexane. 58 g of a yellow powder was obtained. NMR indicated a purity of 95%.

### Example 8

### Preparation of decanoyl vanillin

To a solution of 30.4 g (0.2 mol) of vanillin and 20 g (0.25 mol) of dry pyridine in 500 ml of anhydrous dichloromethane, was added during 30 minutes a solution of 0.2 mol of decanoyl chloride in 75 ml dichloromethane. After addition, the mixture was refluxed for 2 hrs. A white precipitate (pyridine.HCl) was formed. After cooling the precipitate was filtered and the dichoromethane was removed by evaporation. The brown oil was dissolved in 500 ml of toluene and washed three times with 75 ml of brine. Then the solution was washed 3 times with a 5% NaHCO₃ solution. The solvents were removed by evaporation in vacuo. The oil was dissolved in ethyl acetate and stored in the freezer overnight. 45 g (73.5%) of an off-white crystalline product was collected. NMR indicated a purity of 98%.

### Example 9

### Preparation of oleoyl vanillin

To a solution of 30.4 g (0.2 mol) of vanillin and 20 g (0.25 mol) of dry pyridine in 500 ml of anhydrous dichloromethane, was added during 30 minutes an amount of 60.9 g (0.2 mol) of oleoyl chloride in 75 ml dichloromethane. After addition, the mixture was refluxed for 2 hrs. A white precipitate (pyridine.HCl) was formed. After cooling the precipitate was filtered. The filtrate was concentrated by evaporation of the dichoromethane. The brown oil was dissolved in 500 ml of toluene and washed three times with 75 ml of brine. Separation was poor as a oil/water dispersion was formed. Then the toluene solution was washed 3 times with a 5% NaHCO₃ solution. Also here separation between the organic layer and the water layer was poor. The toluene was removed by evaporation to obtain 55 g (68%) of a brown oil. NMR indicated a purity of 90%.

### Example 10

A 5% sucrose solution was prepared and the flavour ingredients prepared in Examples 4-9 were added at 10 ppm concentrations. De solutions were tasted by a trained panel consisting of 5 people. The results are summarised in the following table:

| | Compound | Description |
|---|---|---|
| 1 | Lactoyl isovanillic acid | Sweet, rummy, more body |
| 2 | Lactoyl 4-hydroxy-3- | Rummy, phenolic, full body |
| | methoxy-acetophenone | |
| 3 | Lactoyl 3-hydroxybenzoic | Fruity, alcoholic, more body |
| | acid | |
| 4 | Decanoyl vanilline | Vanilla-like, fatty, slightly rancid |
| 5 | Oleoyl vanilline | Vanilla-like, fatty, sweet, full body, |
| | | complex, lactony |
| 6 | Divanillyl succinate | Vanilla-like, sweet, slightly cooling, |
| | | more body |

### Example 11

### Preparation and Evaluation of propanoyl vanillin

To a solution of 30.4 g (0.2 mol) of vanillin and 20 g (0.2 mol) of triethylamine in 75 ml of chloroform, was added during 30 minutes an amount of 60.9 g (0.2 mol) of propanoyl chloride in 25 ml chloroform. After addition, the mixture was refluxed for 4 hrs. After cooling the chloroform was removed by evaporation. The mixture was solved again in dichloromethane and hexane was added until crystals (triethylamine.HCl) were formed. After cooling the precipitate was filtered. The filtrate was concentrated by evaporation of the solvents. The product had a propanoic acid off-note which was removed by heating at 80° C. at 1 mbar during 15 minutes. 34 g (76%) of final product was obtained. NMR indicated a purity of 98%.

A 5% sucrose solution was prepared and propanoyl vanillin was added in a concentration of 10 ppm. An identical sucrose solution, containing 10 ppm of lactoyl vanillin was prepared for reference. The solutions were tasted by a trained panel consisting of 5 people. The propanoyl vanillin solution was found to have a much weaker flavour than the lactoyl vanillin solution. The propanoyl vanillin solution was described as vanillin-like, but missed the natural vanilla extract character of the lactoyl vanillin solution.

1. A product selected from the group consisting of foodstuffs, beverages, oral care products, tobacco products and flavourings said product containing one or more flavour improving substances selected from the group consisting of compounds represented by the following the formula (I), salts of these compounds and combinations thereof: 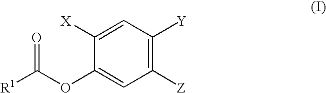
wherein:
X and Z independently represent —H, —OCH₃, —OCH₂CH₃, —CH═O or —COOH;
Y represents —H, —CHO, —COOH, —COCH₃, —CH═CH₂, —CH₂—COOH, —CH₂—CH═CH₂, —CH═CH—CH═O, or —CH═CH—COOH:
at least one of X. Y and Z does not represent —H:
R¹ represents (i) a C₁-C₆ alkyl or C₂-C₆ alkenyl, carrying at least one substituent selected from —OH, —NH₂ and —COOR² and optionally further substituents or (ii) C₇-C₂₁ alkyl, C₇-C₂₁ alkenyl, C₇-C₂₁ alkadienyl or C₇-C₂₁ alkatrienyl, each optionally substituted with one or more substituents selected from —OH, ═O, C₁-C₄ alkoxyl, —COOH and —NH₂;
R² represents —H or a residue represented by the following formula (II): 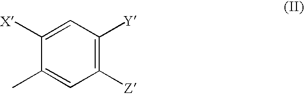
X′, Y′ and Z′ in formula (II) independently have the same meaning as X, Y and Z, respectively in formula (I); and
at least one of X′, Y′ and Z′ does not represent H.

2. Product according to claim 1, wherein, in case the product is a tobacco product or a tobacco flavouring, Y does not represent —CHO.

3. Product according to claim 1, wherein in case the product is a tobacco product R¹ does not represent —COOR² or C₁-C₆ alkyl carrying a substituent selected from —COOR² and —OH.

4. Product according to claim 1, wherein R¹ represents a hydroxyl- or carboxyl-substituted, C₂-C₆ alkyl or C₂-C₆ alkenyl, optionally carrying one or more further substituents selected from hydroxyl and carboxyl.

5. Product according to claim 1, wherein R¹ represents a hydroxyl-substituted, C₂-C₅ alkyl, optionally carrying 1-4 further hydroxyl substituents.

6. Product according to claim 1, wherein R¹ represents a carboxyl-substituted C₂-C₅ alkyl or C₂-C₅ alkenyl, optionally carrying 1-3 further carboxyl substituents.

7. Product according to claim 1, wherein R¹ represents an unsubstituted C₁₅-C₁₇ alkyl or C₁₅-C₁₇ alkenyl, an unsubstituted C₁₅-C₂₁ alkadienyl or an unsubstituted C₁₅-C₂₁ alkatrienyl.

8. Product according to claim 1, wherein X represents —H or —OCH₃.

9. Product according to claim 1, wherein Z represents —CHO, —COOH or —COCH₃.

10. Product according to claim 1, wherein Y represents —H or —COOH.

11. Product according to claim 1, wherein the flavour improving substance is selected from the group of lactoyl vanillin, lactoyl ethylvanillin, lactoyl isovanillin, lactoyl p-hydroxybenzaldehyde, lactoyl p-hydroxy-m-methoxycinnamaldehyde, lactoyl p-hydroxy-m-methoxycinnamic acid, lactoyl vanillic acid, lactoyl homovanillic acid, lactoyl-m-hydroxybenzoic acid, lactoyl isovanillic acid, lactoyl-p-hydroxy-m-methoxyacetophenone, oleyl vanillin, decanoyl vanillin, divanillyl succinate, lactoyl-o-hydroxy-m-methoxybenzaldehyde, lactoyl eugenol, gluconyl vanillin, and edible salts and esters thereof.

12. Product according to claim 1, wherein the composition additionally contains at least 0.5 mg/kg of vanillin.

13. Product according to claim 1, wherein the product is selected from the group consisting of foodstuffs and beverages.

14. Product according to claim 1, wherein the product is a flavouring composition containing at least 0.1 wt. % of one or more flavouring substances and at least 10 mg/kg of the flavour improving substance.

15. Product according to claim 14, wherein the composition contains at least 50 mg/kg of vanillin.

16. A method of preparing a product selected from the group consisting of foodstuffs, beverages, oral care products and tobacco products, said method comprising incorporating into the product at least 0.01 wt. % of a flavouring composition according to claim 14.

17. Method for improving the flavour of a product selected from the group consisting of foodstuffs, beverages, oral care products and tobacco products, comprising incorporating into the product the flavour improving substance as defined in claim 1.

18. A flavour improving substance represented by the following formula (III) or a salt thereof: 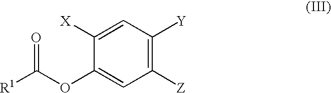
X and Z independently represent —H, —OCH₃, —OCH₂CH₃, —CH═O or —COOH;
Y represents —H, —CHO, —COOH, COCH₃, —CH═CH₂, —CH₂—COOH, —CH₂—CH═CH₂, —CH═CH—CH═O, or —CH═CH—COOH;
at least one of X, Y and Z does not represent —H;
R¹ represents
C₁-C₆ alkyl carrying at least one —NH₂ group and optional further substituents
—CH(OH)CH₃,
—[CH(OH)]₄CH₂(OH),
—CH₂—CH₂—COOR²,
—CH═CH—COOR² or
unsubstituted C₁₅-C₂₁ alkyl, unsubstituted C₁₅-C₂₁ alkenyl, unsubstituted C₁₅-C₂₁ alkadienyl, unsubstituted C₁₅-C₂₁ alkatrienyl, or optionally C₁₅-C₂₁ alkyl, C₁₅-C₂₁ alkenyl, C₁₅-C₂₁ alkadienyl or C₁₅-C₂₁ alkatrienyl substituted with one or more substituents selected from —OH, ═O, C₁-C₄ alkoxyl, —COOH and —NH₂;
R² represents —H or a residue represented by the following formula (IV): 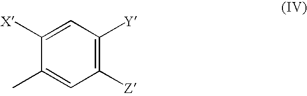
X′, Y′ and Z′ in formula (IV) independently have the same meaning as X, Y and Z, respectively in formula (III); and
at least one of X′, Y′ and Z′ does not represent —H.

19. Flavour improving substance according to claim 18, wherein, in case Y represents —CHO, R¹ does not represent —CH₂—CH₂—COOR².

20. Flavour improving substance according to claim 18, wherein X represents —OCH₃.

21. Flavour improving substance according to claim 18, wherein Z represents —H.

22. Flavour improving substance according to claim 18, wherein Y represents —CHO.

23. Flavour improving substance according to claim 18, wherein R¹ represents —CH(OH)CH₃

24. Flavour improving substance according to claim 18, wherein X represents —OCH₃, Z represents —H, Y represents —CHO, and R¹ represents —CH(OH)CH₃.

25. Flavour improving substance according to claim 18, wherein R¹ represents C₁₇ alkenyl, C₁₇ alkadienyl or C₁₇ alkatrienyl, optionally substituted with one or more substituents selected from —OH, ═O, C₁-C₄ alkoxyl, —COOH and —NH₂.
